(19) United States
(12) Patent Application Publication
(10) Pub. No.: US 2023/0289492 A1
(43) Pub. Date: Sep. 14, 2023

(54) Mixture Modeling Systems and Methods

(71) Applicant: Aka Foods LTD, Tel Aviv (IL)

(72) Inventors: Alex Bronstein, Haifa (IL); David H. Silver, Haifa (IL); Tal Knafo, Haifa (IL); Ariel Harpaz, Mevaseret Zion (IL)

(21) Appl. No.: 17/826,839

(22) Filed: May 27, 2022

Related U.S. Application Data
(63) Continuation-in-part of application No. 17/691,662, filed on Mar. 10, 2022.

(51) Int. Cl. G06F 30/20; G01N 33/02; A47J 44/00

(52) U.S. Cl. CPC G06F 30/20; G01N 33/02; A47J 44/00; G06F 2113/26

(57) ABSTRACT

Example mixture modeling systems and methods are described. In one implementation, a system includes an encoder that receives multiple base ingredients and produces multiple corresponding representations. A composite modeler receives a mixture definition comprising a list of base ingredients and their relative proportions. The composite modeler generates a representation of the mixture. A decoder is receives a representation of a mixture and generates a list of features.

### RELATED APPLICATIONS

This application is a Continuation in Part of U.S. application Ser. No. 17/691,662, entitled “Food Processing Systems and Methods,” filed Mar. 10, 2022, the disclosure of which is incorporated herein by reference in its entirety.

### TECHNICAL FIELD

The present disclosure relates to systems and methods to create and test food products using a variety of ingredients at the molecular level.

### BACKGROUND

Existing techniques for creating new food products and associated recipes often require significant experimentation and considerable human tasting. Additionally, these existing techniques may require an experienced chef or other food product designer to create new combinations of ingredients that are likely to taste good to a human.

The techniques that require an experienced chef, significant experimentation, and many human tasting tests can be expensive and time-consuming. Further, those techniques can be limited to the chef's personal experience with different types of recipes and ingredients. The need exists for systems and methods that can create new food products and develop new recipes in a manner that accesses a wider universe of ingredients, is less expensive, and requires less trial-and-error to implement.

### BRIEF DESCRIPTION OF THE DRAWINGS

Non-limiting and non-exhaustive embodiments of the present disclosure are described with reference to the following figures, wherein like reference numerals refer to like parts throughout the various figures unless otherwise specified.

FIG. 1 is a block diagram illustrating an environment within which an example embodiment may be implemented.

FIG. 2 is a flow diagram illustrating an embodiment of a process for preparing and testing new preparation instructions.

FIG. 3 is a block diagram illustrating an embodiment of a process flow for predicting characteristics of preparation instructions.

FIG. 4 is a block diagram illustrating an embodiment of a process flow for optimizing creation of new preparation instructions.

FIG. 5 is a block diagram illustrating an embodiment of a mixture modeler.

FIG. 6 is a block diagram illustrating an embodiment of a process flow for an inference mode of operation.

FIG. 7 is a flow diagram illustrating an embodiment of a process for inferring features of a mixture of base ingredients.

FIG. 8 is a block diagram illustrating an embodiment of a process flow for a training mode of operation.

FIG. 9 is a flow diagram illustrating an embodiment of a process for training a mixture modeler.

FIG. 10 is a block diagram illustrating an embodiment of a process flow for an inverse mode of operation.

FIG. 11 is a flow diagram illustrating an embodiment of a process for implementing an inverse mode of operation.

FIG. 12 illustrates an example block diagram of a computing device.

### DETAILED DESCRIPTION

The perception of taste is a psychological experience based primarily on the structural and chemical molecular properties of various ingredients and their interactions with the taste and smell receptors and themselves.

In some embodiments, the systems and methods discussed herein identify the objective properties of ingredients and molecules. The objective properties of various ingredients and molecules are translated into a subjective tasting experience that may include, for example, its savor, smell, texture, and mouthfeel.

As discussed herein, the described systems and methods can evaluate actual human tasting results as well as the objective properties using a food processing unit (FPU) and other components or systems to generate reliable distributions of predicted user responses from a small number of actual human tastings. Thus, the systems and methods may provide alternate materials or ingredients that can be mixed and prepared to provide tasting experiences similar to traditional foods with minimal human tasting activities.

In some embodiments, the described systems and methods may identify new ingredients and preparation instructions for traditional foods that eliminate animal products, eliminate certain food allergens, replace expensive ingredients, replace ingredients that are in short supply, and the like.

In the following disclosure, reference is made to the accompanying drawings, which form a part hereof, and in which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Implementations of the systems, devices, and methods disclosed herein may comprise or utilize a special purpose or general-purpose computer including computer hardware, such as, for example, one or more processors and system memory, as discussed herein. Implementations within the scope of the present disclosure may also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations of the disclosure can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

An implementation of the devices, systems, and methods disclosed herein may communicate over a computer network. A “network” is defined as one or more data links that enable the transport of electronic data between computer systems and/or modules and/or other electronic devices. When information is transferred or provided over a computer network or another communications connection (either hardwired, wireless, or a combination of hardwired or wireless) to a computer, the computer properly views the connection as a transmission medium. Transmission media can include a computer network and/or data links, which can be used to carry desired program code means in the form of computer-executable instructions or data structures and which can be accessed by a general purpose or special purpose computer. Combinations of the above should also be included within the scope of computer-readable media.

Computer-executable instructions comprise, for example, instructions and data which, when executed at a processor, cause a general purpose computer, special purpose computer, or special purpose processing device to perform a certain function or group of functions. The computer executable instructions may be, for example, binaries, intermediate format instructions such as assembly language, or even source code. Although the subject matter is described in language specific to structural features and/or methodological acts, it is to be understood that the subject matter defined in the appended claims is not necessarily limited to the described features or acts described herein. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by a combination of hardwired and wireless data links) through a communication network, both perform tasks. In a distributed system environment, program modules may be located in both local and remote memory storage devices.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the description and claims to refer to particular system components. As one skilled in the art will appreciate, components may be referred to by different names. This document does not intend to distinguish between components that differ in name, but not function.

It should be noted that the sensor embodiments discussed herein may comprise computer hardware, software, firmware, or any combination thereof to perform at least a portion of their functions. For example, a sensor may include computer code configured to be executed in one or more processors, and may include hardware logic/electrical circuitry controlled by the computer code. These example devices are provided herein for purposes of illustration, and are not intended to be limiting. Embodiments of the present disclosure may be implemented in further types of devices, as would be known to persons skilled in the relevant art(s).

At least some embodiments of the disclosure are directed to computer program products comprising such logic (e.g., in the form of software) stored on any computer useable medium. Such software, when executed in one or more data processing devices, causes a device to operate as described herein.

Various terms are used in this specification to describe systems, methods, ingredients, molecular structures, processing steps, data, and the like. For example, the following terms are briefly described for a particular embodiment. It should be understood that the following descriptions are presented by way of example only, and not limitation. It will be apparent to persons skilled in the relevant art that different descriptions may be provided for these terms without departing from the spirit and scope of the disclosure.

In some embodiments, vectors are represented as bold, roman, lower case. Scalars may be represented as italics. Matrices may be represented as bold, roman, upper case. T may represent transpose.

The systems and methods define the fundamental and indivisible constituent of a food product as a simple (mono-molecular) ingredient—a substance containing only one type of molecules. Simple ingredients may be mixed in some proportions forming composite ingredients. Ingredients may be further subjected to various types of transformations such as heating or cooling. From this perspective, any food product can be described as a sequence of mixing and transformation operations applied initially to the raw simple ingredients, and to the intermediate products until the final product is obtained. We refer to such a sequence as “preparation instructions”, while the list of the initial ingredients and their quantities is referred to as the “formula” of the food product.

We henceforth refer to the set of characteristics of a food product pertaining to the flavor perception it generates as to its “flavor profile”. A flavor profile may contain objective characteristics characterizing the sensory response (for example, the binding affinities of the different molecular constituents of the food product to a set of known taste receptor proteins, or mechanical properties such as elasticity as a function of temperature), as well as subjective characteristics (for example, a verbal description of the food product's taste and smell and its comparison to other reference products in the sense of some fixed flavor features such as sweetness, bitterness, sourness, texture, and mouthfeel).

In some embodiments, an ingredient is a natural or synthetic mixture of molecules in some concentrations (e.g., relative amounts). An ingredient can be simple (mono-molecular) or composite (comprising more than one molecule). Concentrations and the constituent molecules of an ingredient can be determined by chemical analytic methods such as liquid chromatography (LC) and mass spectrometry (MS).

A formula may include a list of ingredients with their quantities, which is different from a chemical formula. A mixture is the result of mixing various ingredients according to a formula. The chemical composition of a mixture may change based on chemical reactions between the constituent molecules.

A transformation is an operation or process applied to an ingredient, such as baking at 180 degrees Celsius for 5 minutes.

A preparation instruction is a directed graph starting from a formula and applying a sequence of mixtures and transformations resulting in a single output food (prepared according to the preparation instructions). In some embodiments, a food may also be an ingredient.

A subjective flavor profile may include a description of how the taste/smell of an ingredient is perceived by a human taster. It may also include one or more keywords that approximate the evoked perception, a vector of scores numerically grading different flavor features (sweetness, bitterness, and the like), or a comparison of the above features to another ingredient (e.g., A is sweeter than B, A is more bitter than C, A is as sour as D, and the like).

An objective flavor profile may include measurable physical and chemical characteristics such as pH, viscosity, hardness, and the like.

A flavor profile may be a combination of the subjective flavor profile and the objective flavor profile.

In some embodiments, the systems and methods described herein may receive an ingredient list and a reference food. Based on the ingredient list and reference food, the systems and methods generate preparation instructions for a particular food item using one or more alternate ingredients than the traditional preparation instructions.

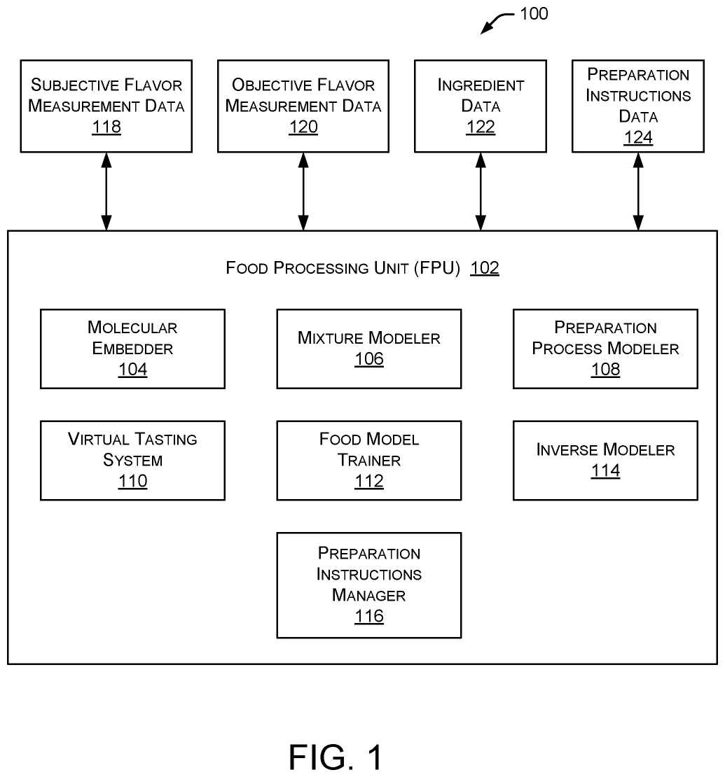

FIG. 1 is a block diagram illustrating an environment 100 within which an example embodiment may be implemented. As shown in FIG. 1, environment 100 includes a food processing unit (FPU) 102 that may implement one or more processes to replicate food characteristics using a mixture of food items, ingredients, and the like. In some embodiments, FPU 102 may replicate food characteristics based on information (e.g., data-driven elements) stored in one or more databases, as discussed herein.

In some implementations, FPU 102 may contain or access a digitization of one or more food features using various combinations of subjective food tastings, mixture prediction, and molecule taste prediction. FPU 102 then generates new preparation instructions that are similar to the food to be created. A profile of the food to be created may be generated from the subjective food tastings, analytical data (e.g., liquid chromatography mass spectrometry), and other information discussed herein.

As shown in FIG. 1, FPU 102 includes a molecular embedder 104 capable of performing a molecular embedding process. The molecular embedding process can produce a representation of the chemical and structural information of a mono-molecular tastant substance from which its flavor profile can be predicted. A tastant substance is any substance capable of producing a taste sensation (e.g., eliciting gustatory and/or olfactory excitation).

In some embodiments, molecular embedder 104 is implemented as a learned model that conceptually follows an auto-encoder architecture. The input to the encoder model is a molecular profile that includes the molecular structure and its chemical and physical properties, which is collectively denoted by the vector m. The output of the encoder model is a latent vector z=E(m). A decoder D is a learned model that receives a latent vector z representing the mono-molecular tastant substance and predicting a property of the mono-molecular tastant substance.

In some embodiments, multiple decoding heads are used, such as:

1. Dₐᵤₜₒ≈E⁻¹−A model predicting the molecular profile vector m itself. The model ensures that Dₐᵤₜₒ∘E≈Id makes the latent representation complete about the input molecule.

2. Dₛₑₙₛ−A model predicting the sensory response of certain gustative and olfactory receptor cells.

For simplicity of explanation, when describing the systems and methods herein, the explanation may refer to the encoder model as a deterministic one. A specific embodiment may instead represent, in some parametric form, the distribution of E(m) in the latent space.

As illustrated in FIG. 1, FPU 102 also includes a mixture modeler 106 capable of producing a representation of composite tastants that include multiple molecules. In some embodiments, mixture modeler 106 is a learned embedding model that receives an unordered collection Z={z₁, z₂, . . . , zₙ} of a fixed arbitrary number n of molecular embeddings. Mixture modeler 106 also receives a vector α=(α₁, α₂, . . . , αₙ) on the probability simplex representing their relative quantities in the mixture. Mixture modeler 106 produces an output that is another latent vector w=M(Z, α). For simplicity and based on a simple transitivity property, the following discussion assumes n=2, such that w=M(z₁, z₂, α, 1−α).

In some implementations, mixture modeler 106 is built to approximately satisfy homogeneity and additivity under the mixture, such as:

M(z₁, z₂, α, 1−α)=αM(z₁)+(1−α)M(z₂)

In some embodiments, for purposes of convenience, the coordinate system is defined such that water is represented as zero.

In particular implementations, using mixture modeler 106 and asserting one of the mixands to be a solvent (e.g., water), the systems and methods can define another decoder head operating on the mixture representation space:

D_{subj}−A model predicting the subjective flavor profile. For example, in the case of a molecule m at concentration α in water, D_{subj}(αM∘E(m))=f predicts the perceived flavor characteristics, such as flavor categories, flavor feature scores, and relations to reference flavors, which are collectively denoted by the (pseudo-) vector f.

In some embodiments, the described systems and methods may assert the same space suiting both mono-molecular and mixture embeddings. In these implementations, the systems and methods use z and M(z) interchangeably (e.g., referring to both as z), such that the systems and methods may assume M∘E in place of E.

In some embodiments, FPU 102 further includes a preparation process modeler 108 capable of representing the effect of cooking and preparation processes on the latent representation. In some situations, a preparation process model may also be referred to as a precision graph or cooking graph.

In particular implementations, preparation process modeler 108 models a single step of the preparation process as a transformation of the latent space T(w)=w′. Using these terms, preparation instructions can be thought of as the composition of binary mixture and unary preparation operations. For example:

T₂(M(T₁(M(z₁, z₂, α)), z₃, α′)=T₂(α′T₁(αz₁+(1−α)z₂)+(1−α′)z₃).

In some embodiments, such a sequence can be represented as a tree with basic mono-molecular ingredients as the leaf nodes and the final food product at the root. The ingredients themselves, Z=(z₁, z₂, . . . , zₙ) and their relative quantities α=(α₁, α₂, . . . , αₙ) can be referred to as the formula of the food, which is different from the chemical formula. In some implementations, preparation instructions may be represented using a shorthand notation T(Z, α).

As shown in FIG. 1, FPU 102 also includes a virtual tasting system 110 capable of providing a virtual tasting room for testing new food products, food ingredients, and the like. In some embodiments, virtual tasting system 110 can predict which users may like a particular food product and which users may be the best testers of new food products or testing two or more similar food products.

Virtual tasting system 110 may support food testing and obtaining feedback on new food products using a smaller group of human testers. Instead of testing food products with a large number of random people, virtual tasting system 110 can provide valuable feedback on new food products using a smaller number of human testers. For example, the human testers for a new food product may be selected based on the human testers' food preferences, previous tasting event results, and the like.

In some embodiments, a tasting event produces various results that may include data related to taster preferences for one or more food products or compounds. Based on these results, each taster's profile may be updated based on their tasting preferences, and each food product's profile may be updated based on the tasting results from multiple tasters.

In some embodiments, virtual tasting system 110 may implement graph learning methods by, for example, predicting a taster's response to a substance. Based on sparse data collected from multiple tasters related to multiple substances, a deep neural network may be trained that recreates the geometry of the taster's space (e.g., the intra tasters relations) and the geometry of the substance space (e.g., the intra substance relations). Additionally, the deep neural network may be trained to recreate the interrelation between the tasters' graph and the substances' graph. In some embodiments, virtual tasting system 110 also supports the generation of new tasters, based on the required demographic and other background questionnaires, and prediction of the new tasters' response to a variety of substances.

In some embodiments, FPU 102 further includes a food model trainer 112 capable of training food models using a multi-task learning approach. In some implementations, individual models (e.g., molecular embedding models, mixture models, and preparation process models) can be pre-trained using individual sets of tasks followed by joint fine-tuning. Example learning tasks may include the following:

1. Homogeneity: asserting that given mixtures of ingredients z₁, z₂ in concentrations α, 1−α, and the flavor profile f of the mixture:

D_{subj}(αz₁+(1−α)z₂)=f

2. Transformed flavor profile: given pairs of flavor profiles (f, f′) of ingredients before and after a certain preparation process (e.g., heating to 180 degrees Celsius for 15 minutes), the transformed model can be trained by minimizing the discrepancy of the predicted taste profiles, D_{subj}∘T(f) and D_{subj}(f′).

3. Transformed chemistry: given pairs of chemical compositions ((Z, α), (Z′, α′)) of ingredients before and after a certain preparation process, the transformation model can be trained by minimizing the discrepancy of the predicted molecular profiles, T(α₁z₁+ . . . +αₙzₙ) and α′₁z′₁+ . . . +α′_{n′}z′_{n′}.

As shown in FIG. 1, FPU 102 also includes an inverse modeler 114, which takes the approach of designing a food product in an inverse manner. For example, inverse modeler 114 may solve an inverse problem that includes finding a formula having n molecular ingredients M=(m₁, m₂, . . . , mₙ), their quantities α, and the preparation process T(E(M), α). Inverse modeler 114 attempts to satisfy the following list of constraints. In particular embodiments, some or all of the constraints can be equivalently cast as optimization objectives.

1. Number of ingredients

2. Similarity to a target flavor profile D_{subj}∘T(E(M), α)=f_{target}, where f_{target} denotes the target flavor profile

3. Nutritional values of the molecular ingredients

4. Product cost including the sum of the cost of each mᵢ weighted by αᵢ and, in some situations, by the cost of all preparation stages comprising T.

The solution of the inverse problem can be carried out using regular backpropagation techniques.

In the case where the encoder E is stochastic, rather than getting a single solution, the systems and methods produce a posterior distribution from which multiple solution candidates can be sampled.

In some embodiments, FPU 102 further includes a preparation instruction manager 116 capable of storing and managing various preparation instructions. For example, preparation instruction manager 116 may track various ingredients, mixture ratios, and processing steps for different preparation instructions. Additionally, preparation instruction manager 116 may record tasting results (both subjective and objective) for various preparation instructions so the data can be used for creating different preparation instructions in the future. Preparation instruction manager 116 may also monitor and record visual, mechanical, and chemical properties of the prepared food.

In some embodiments, environment 100 further includes subjective flavor measurement data 118, objective flavor measurement data 120, ingredient data 122, and preparation instruction data 124. Subjective flavor measurement data 118 may include subjective results associated with an ingredient or preparation instruction by a human user. For example, subjective flavor measurement data 118 may include human user opinions regarding taste, texture, odor, and the like for a particular preparation instruction or ingredient.

In some embodiments, objective flavor measurement data 120 includes objective results associated with an ingredient or preparation instruction by a human user. For example, objective flavor measurement data 120 may include objective flavor profile data that is created or predicted using the systems and methods described herein. The objective flavor profile data may include predicted data regarding taste, texture, odor, and the like for a particular preparation instruction or ingredient.

Ingredient data 122 may include information associated with particular ingredients, such as an ingredient flavor profile, taste testing results associated with the ingredient, preparation instructions that include the ingredients, and the like. Preparation instruction data 124 may include information associated with various preparation instructions. In some embodiments, preparation instruction data 124 includes preparation instruction ingredients, preparation instruction mixing instructions, preparation instruction process, preparation instruction flavor profiles, preparation instruction taste testing results, and the like.

In some embodiments various ingredient data and preparation instruction data may be accessed or received from public databases combined with a measured outcome (e.g., objective or subjective features). In some implementations, the systems and methods described herein may perform pairwise comparisons or absolute taste grades with respect to different features, flavor keywords, and the like. In the case of absolute taste grades, the systems and methods may add heads that predict those characteristics.

It will be appreciated that the embodiment of FIG. 1 is given by way of example only. Other embodiments may include fewer or additional components without departing from the scope of the disclosure. Additionally, illustrated components may be combined or included within other components without limitation.

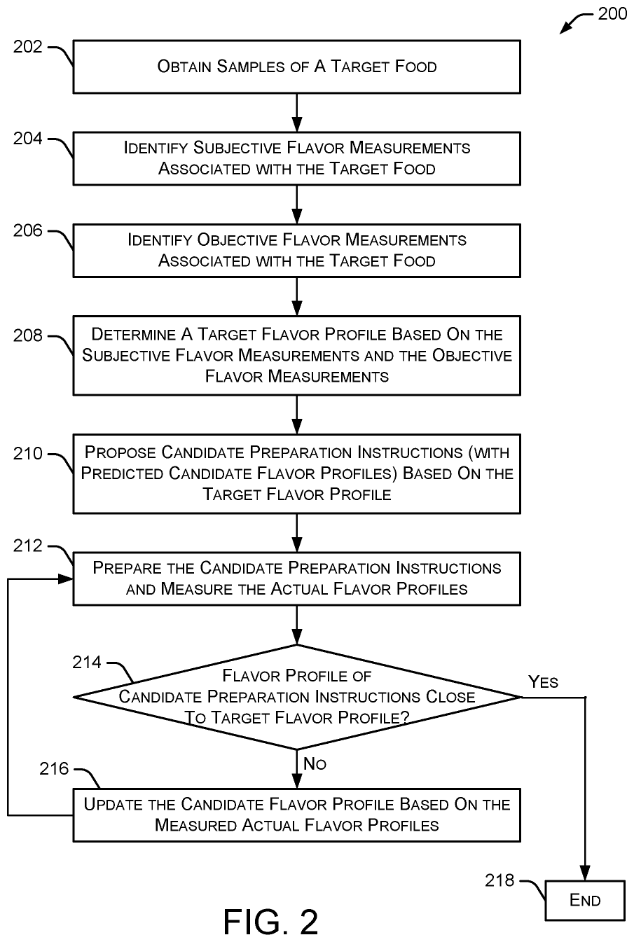

FIG. 2 is a flow diagram illustrating an embodiment of a process 200 for preparing and testing new preparation instructions. Initially, process 200 obtains 202 samples of a target food. In some embodiments, a target food may be a traditional food that is being copied by creating new preparation instructions with different ingredients, but a similar flavor profile. For example, the target food may be a traditional food that includes one or more animal-based ingredients. The systems and methods described herein are used to prepare a new version of the traditional food without animal-based ingredients, but maintaining the traditional food's flavor profile.

Process 200 continues by identifying 204 subjective flavor measurements associated with the target food. For example, the subjective flavor measurements may include taste, texture, smell, and the like. In particular implementations, the subjective flavor measurements are based on responses from human users who tasted the target food.

Process 200 then identifies 206 objective flavor measurements associated with the target food. For example, the objective flavor measurements may include physical and chemical information that may be used to predict taste, texture, smell, and the like. In some embodiments, the objective flavor measurements may be obtained as predictions from virtual tasting system 110 and other components of FPU 102.

The process continues by determining 208 a target flavor profile based on the subjective flavor measurements and the objective flavor measurements. This target flavor profile is used to create new preparation instructions with the same, or similar, flavor profiles as the existing food product. Process 200 then proposes 210 one or more candidate preparation instructions with predicted candidate flavor profiles based on the target flavor profile. In some embodiments, the candidate preparation instructions are expected to have predicted candidate profiles that are close to the target flavor profile.

The process continues by preparing 212 the one or more candidate preparation instructions and measuring the actual flavor profiles of the candidate preparation instructions. The process then compares the actual flavor profiles of the candidate preparation instructions to the target flavor profile. Process 200 continues by determining 214 whether the actual flavor profiles of the candidate preparation instructions are close to the target flavor profile. If the actual flavor profiles of the candidate preparation instructions are close to the target flavor profile, the process ends at 218. In some embodiments, the candidate preparation instructions that are close to the target flavor profile may be tested by one or more human users to determine whether the flavor of the food product created with one or more candidate preparation instructions is a viable replacement for the target food.

If the actual flavor profiles of the candidate preparation instructions are not close to the target flavor profile, process 200 updates 216 the candidate flavor profile based on the measured actual flavor profiles. The process then returns to 212, where the updated candidate preparation instructions are prepared and their actual flavor profiles are measured. The process further determines whether the actual flavor profiles of the updated candidate preparation instructions are close to the target flavor profile. This process of updating candidate preparation instructions and determining updated actual flavor profiles is repeated until the flavor profile of one or more candidate preparation instructions is close to the target flavor profile.

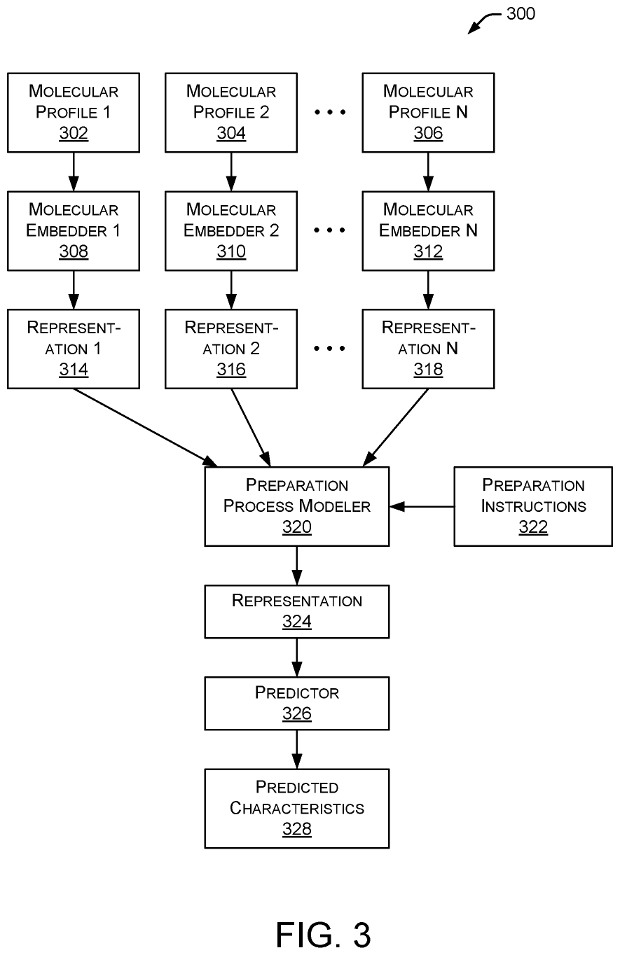

FIG. 3 is a block diagram illustrating an embodiment of a process flow 300 for predicting characteristics of a particular preparation instruction. As shown in FIG. 3, process flow 300 receives multiple molecular profiles 302, 304, and 306. Each molecular profile 302-306 defines various properties of a molecule or molecular structure that may be included in the results of a preparation instruction or other mixture. The molecular profiles 302-306 are provided to a molecular embedder 308, 310, 312, respectively. Molecular embedders 308-312 may be similar to molecular embedder 104 shown in FIG. 1 and discussed herein.

In process flow 300, each molecular embedder 308, 310, 312 generates a representation 314, 316, 318, respectively. Representations 314-318 of each molecule are vectors created via a (trainable) non-linear map of the input data. Each representation 314-318 contains enough dimensions such that the corresponding decoder heads can extract the required information with sufficient precision.

In some embodiments, the representations 314-318 are provided to a preparation process modeler 320. Preparation process modeler 320 may be similar to preparation process modeler 108 shown in FIG. 1 and discussed herein. Preparation process modeler 320 also receives preparation instructions 322, which may describe how the multiple molecular profiles 302-306 are mixed and processed.

Preparation process modeler 320 receives the representations of the input ingredients and generates a representation 324 of the prepared ingredient.

In some embodiments, representation 324 is provided to a predictor 326. Predictor 326 represents decoder heads that extract different objective and subjective characteristics from the representation vector regarding the food product being represented. In some embodiments, predictor 326 generates any number of predicted characteristics 328 related to the food product associated with representation 324. For example, predicted characteristics 328 may include a flavor profile associated with the food product identified in representation 324.

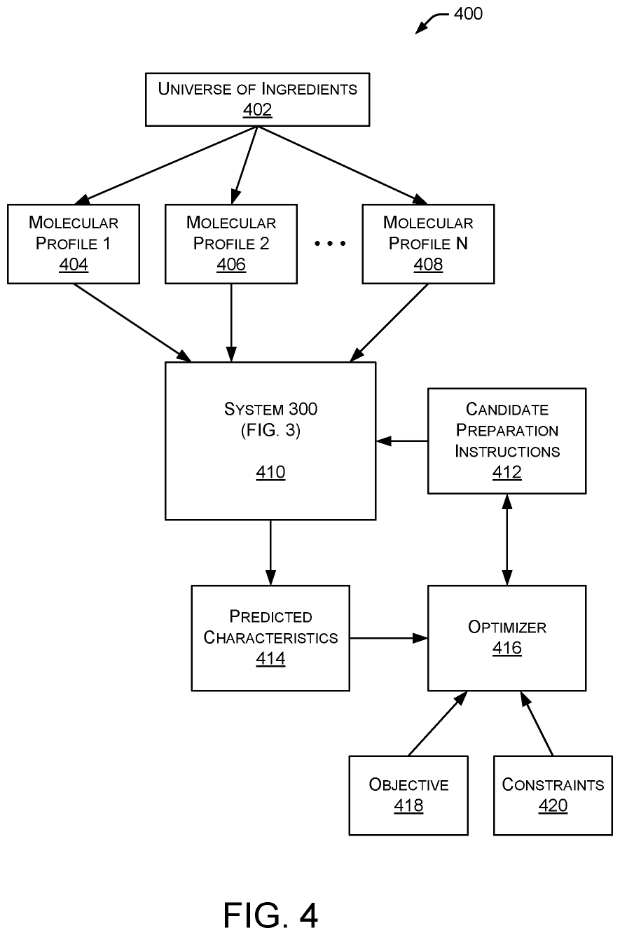

FIG. 4 is a block diagram illustrating an embodiment of a process flow 400 for optimizing creation of new preparation instructions. In the example of FIG. 4, one or more molecular profiles 404, 406, and 408 are selected from a universe of ingredients 402. Each molecular profile 404-408 defines various properties of a molecule or molecular structure that may be included in a preparation instruction or other mixture. The molecular profiles 404-408 are provided to a system 410 of the type shown in FIG. 3.

In some embodiments, system 410 receives a list of ingredients 404-408 and instructions about their preparation 412 (e.g., preparation instructions), then predicts a set of characteristics 414 of the final food product. Optimizer 416 may decide how to modify the candidate preparation instructions 412 to better match the objective or constraints. In some implementations, system 410 is the forward model that is inverted in the inverse modeler.

As shown in FIG. 4, system 410 generates candidate preparation instructions 412. System 410 also communicates predicted characteristics 414 to an optimizer 416. Optimizer 416 may also receive candidate preparation instructions 412. Optimizer 416 also receives objective information 418 and constraints information 420. In some embodiments, objective information 418 and constraints information 420 may be used by optimizer 416 to optimize a particular recipe. In particular implementations, optimizer 416 is part of FPU 102 working in the inverse mode (e.g., proposing new preparation instructions). For example, optimizer 416 may optimize the preparation instructions.

In the forward mode, given preparation instructions, the systems and methods described herein predict the characteristics of the preparation instructions. In the inverse mode, given particular target characteristics, the systems and methods find preparation instructions that satisfy the target characteristics.

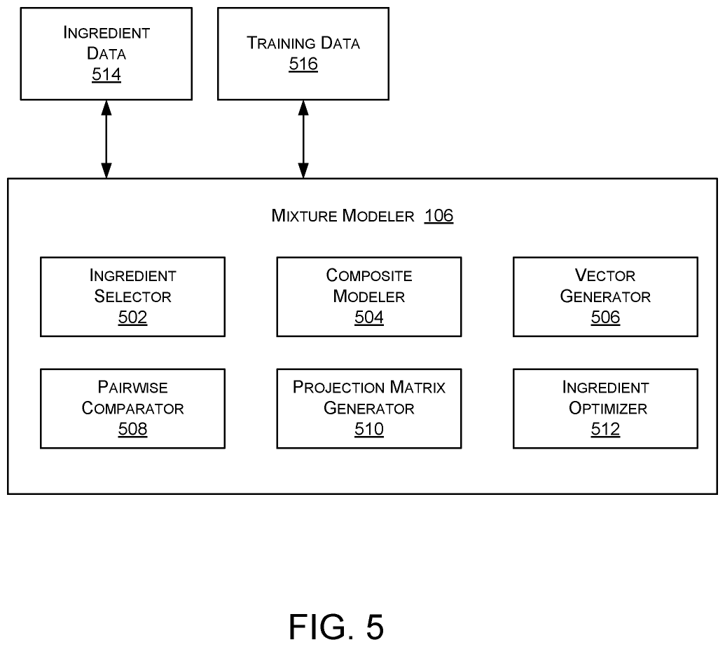

FIG. 5 is a block diagram illustrating an embodiment of mixture modeler 106. As discussed above, some embodiments of mixture modeler 106 are capable of producing a representation of composite tastants that include multiple molecules. In particular implementations, mixture modeler 106 is a learned embedding model M that receives an unordered collection Z={z₁, z₂, . . . , zₙ} of an arbitrary number n of molecular embeddings. Mixture modeler 106 also receives a vector α=(α₁, α₂, . . . , αₙ) on the probability simplex representing their relative quantities in the mixture. Mixture modeler 106 produces an output that is another latent vector w=M(Z, α).

As shown in the example of FIG. 5, mixture modeler 106 may include an ingredient selector 502, a composite modeler 504, a vector generator 506, a pairwise comparator 508, a projection matrix generator 510, and an ingredient optimizer 512. As discussed herein, ingredient selector 502 may select any number of ingredients for testing or evaluating. In some embodiments, ingredient data 514 includes information related to a variety of different ingredients, molecules, and the like. Ingredient selector may select one or more ingredients based on ingredient data 514 as well as other ingredient information from any data source. Additionally, mixture modeler 106 may access training data 516 when implementing any of the functions or activities discussed herein.

In some embodiments, composite modeler 504 may receive a mixture definition that includes a list of base ingredients and their relative quantities. Composite modeler 504 may output a representation of a particular mixture. As discussed herein, vector generator 506 may generate a vector having multiple dimensions that represent features associated with a mixture. The features may include, for example, a taste, a smell, a texture, or a nutritional value associated with the mixture.

In particular implementations, pairwise comparator 508 may receive a pair of feature lists (e.g., from two different mixtures) and produces a list of pairwise comparisons based on the pair of feature lists. The pairwise comparator 508 may also determine if one of the mixtures has a stronger presence of a feature than the other mixture. In some embodiments, projection matrix generator 510 may support handling of cases in which not all measurements are given or when the measurements are performed in a different basis. As discussed herein, ingredient optimizer 512 may optimize any number of ingredients in a mixture to achieve the desired results, such as desired taste, desired smell, desired texture, desired nutritional value, and the like.

In some embodiments, the purpose of mixture modeling (MM) is to produce a representation of composite tastants comprising multiple molecules. For example, define a universe of n base ingredients and refer to them by their index, i=1, . . . , n. Base ingredients can be mixed together in arbitrary proportions to produce new composite ingredients. A mixture will be represented by an n-dimensional vector α=(α₁, . . . , αₙ) on the probability simplex (i.e., having non-negative entries summing to 1, and representing the relative quantity of each base ingredient in the mixture. Using this notation, the base ingredients can be represented by the standard base vectors, e₁, . . . , eₙ (where each eₖ has 1 in coordinate k and zeros elsewhere).

In some embodiments, more complicated preparation process can be applied to the base ingredients. In that case, the process can be represented as a directed tree-structured graph with the base ingredients on its leaf nodes. A subset of nodes can be mixed in proportions specified on the graph edges, producing a new node representing the composite tastant. A node representing a tastant can also undergo processing like heating or cooling, producing a new node representing the product tastant. In the latter case, the type of processing and its parameters are encoded as edge attributes connecting the two nodes. The root of the tree represents the final product of the preparation process.

Some embodiments further define a set of m features measured objectively (e.g., quantitative sensory response to an ingredient) or subjectively (e.g., the sweetness or sourness of an ingredient). Given a pair of ingredients represented by mixture coefficients α and α′, a pairwise comparison can determine how the first ingredient is compared to the second ingredient in terms of each of the features. These measurements may be represented as an m-dimensional vector y∈{−1, 0, 1}^{m}, where +1 in coordinate k means that the first ingredient is “bigger” than the second ingredient in the sense of feature k (e.g., if feature k represents sweetness, then +1 implies that the first ingredient is sweeter); similarly, −1 implies the reverse, and 0 means that the two ingredients are about the same with respect to feature k.

Sometimes, not all features may be measured. To model such partial measurement situations, some embodiments may use a projection matrix P defining a subspace of features where the measurements are available (for all measurements, P is set to the identity matrix).

In some embodiments, combinations of features may be measured instead of “pure” values of each individual feature. To model such superposition measurements, some embodiments may use a projection matrix P defining the measurement operator.

In some embodiments, a non-linear transformation of features may be measured instead of features or their combinations. The projection P in such cases should be interpreted as a general known non-linear map.

In some embodiments, the goal is to create a representation of the base ingredients in an m-dimensional embedding space, such that each ingredient is modeled by a vector x. In some implementations, the following properties may be satisfied by a good representation:

1. Order relation: Let a pair of ingredients represented by x and x′, respectively, be compared producing a pairwise comparison vector y. Then, xₖ>yₖx′ₖ if yₖ=±1 and xₖ≈x′ₖ if yₖ=0, for each k=1, . . . , m.

2. Homogeneity: A mixture of ingredients represented by x and x′ should be represented by αx+(1−α)x′, with α and 1−α being the mixing proportions of the first and the second ingredients, respectively.

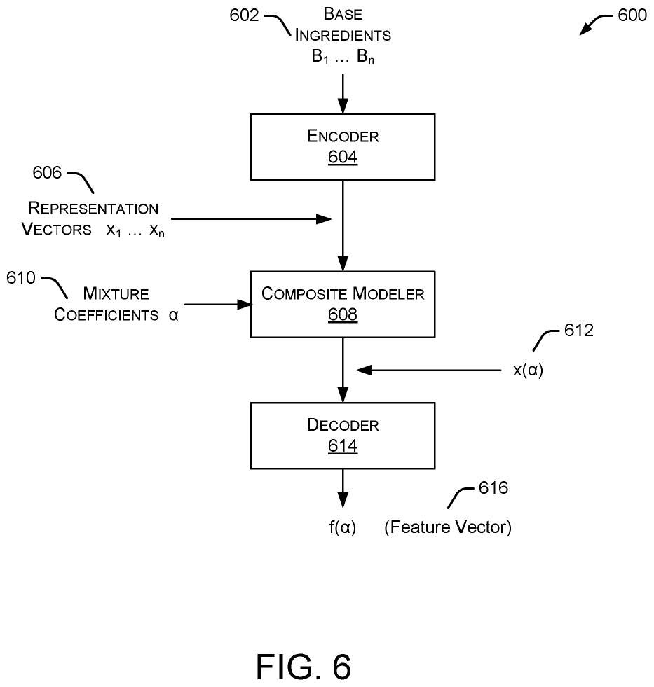

FIG. 6 is a block diagram illustrating an embodiment of a process flow 600 for an inference mode of operation. As shown in FIG. 6, any number of base ingredients 602 (B₁, B₂, . . . B_{N}) are provided to an encoder 604, which generates representation vectors 606 (x₁, x₂, . . . x_{N}) based on the received base ingredients 602. Representation vectors 606 are provided to a composite modeler 608, which also receives mixture coefficients 610 (α). Mixture coefficients 610 are vectors that have n dimensions and identify a quantity of each base ingredient in the mixture. Composite modeler 608 outputs a representation of the mixture x(α) 612 with coefficients a. The output of composite modeler 608 is provided to a decoder 614, which generates a feature vector f(α) 616. The feature vector 616 may indicate a particular feature of the mixture, such as taste, smell, texture, a nutritional value, and the like. Thus, the process 600 receives one or more base ingredients and their relative quantities, represents the mixture as a vector, and infers expected features of the mixture.

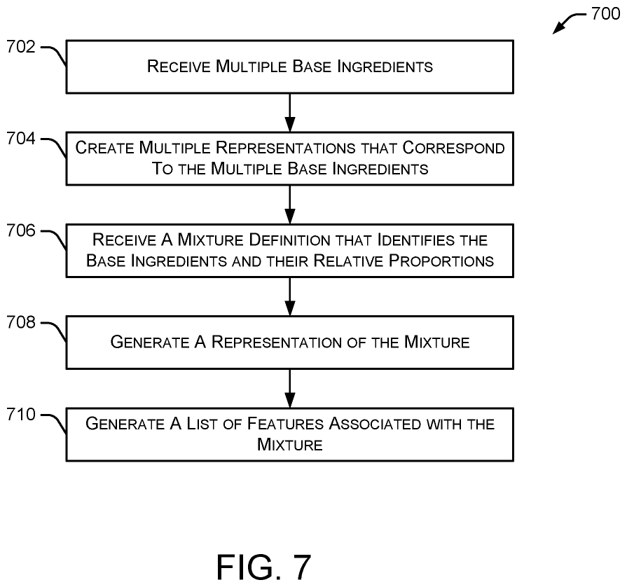

FIG. 7 is a flow diagram illustrating an embodiment of a process 700 for inferring features of a mixture of base ingredients. In the example of FIG. 7, the process receives 702 multiple base ingredients and creates 704 multiple representations that correspond to the multiple base ingredients. Process 700 receives 706 a mixture definition that identifies the base ingredients and their relative proportions. The process then generates 708 a representation of the mixture and generates 710 a list of features associated with the mixture. As discussed herein, the features may include smell, taste, texture, nutritional information, and the like.

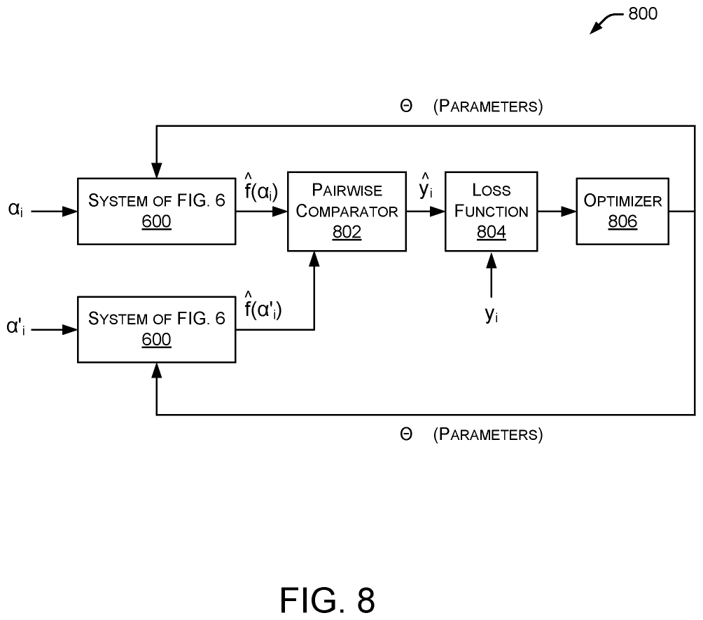

FIG. 8 is a block diagram illustrating an embodiment of a process flow 800 for a training mode of operation. As shown in FIG. 8, two instances of the system 600 shown in FIG. 6 receive mixture coefficients αᵢ and α′ᵢ. The two instances of the system 600 also receive the same learned parameters (θ). The outputs of the two instances of system 600, f(αᵢ) and f(α′ᵢ), represent feature vectors that predict the features of the mixture coefficients processed by system 600. The feature vectors \hat{f}ᵢ(αᵢ) and \hat{f}ᵢ(α′ᵢ) are communicated to a pairwise comparator 802, which compares the two feature vectors and generates a vector ŷᵢ that predicts whether a particular feature is stronger in αᵢ or α′ᵢ (or whether the particular feature is about the same in both αᵢ and α′ᵢ). As shown in FIG. 8, vector ŷᵢ is communicated to a loss function 804, which also receives ground truth information ŷᵢ that is generated based on human tasting, mechanical properties, and the like. The output of loss function 804 is provided to optimizer 806 which adjusts one or more parameters (θ) to minimize the loss function. The adjusted parameters (θ) are then communicated back to the two instances of the system 600, which represents an iteration operation.

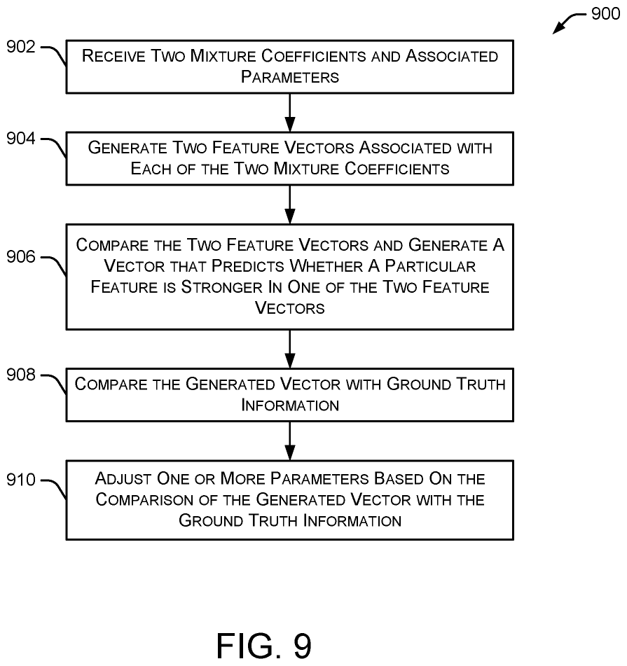

FIG. 9 is a flow diagram illustrating an embodiment of a process 900 for training a mixture modeler, such as mixture modeler 106 discussed herein. Initially, process 900 receives 902 two mixture coefficients and associated parameters. The process generates 904 two feature vectors associated with each of the two mixture coefficients. Process 900 continues by comparing 906 the two feature vectors and generates a vector that predicts whether a particular feature is stronger in one of the two feature vectors. Process 900 then compares 908 the generated vector with ground truth information. One or more parameters are adjusted 910 based on the comparison of the generated vector with the ground truth information. The adjusted parameters may then be used in an iterative process that predicts ŷᵢ, compares the ŷᵢ values, evaluates the loss (and its gradients), updates the parameters, and repeats the process until convergence.

The following discussion describes the construction of the embedding from training data. As the input, the described systems and methods receive a collection of tuples of the form:

{αᵢ, α′ᵢ, Pᵢyᵢ}ᵢ₌₁^{N}

The learnable degrees of freedom are the embeddings of the base ingredients. In order to account for uncertainty in the latter, some embodiments represent each base ingredient k by a parametric m-variate probability distribution, p_{θ}_{ₖ}(x), where the set of parameters θ={θ₁, . . . , θₙ} are the learned variables. In some implementations, the family of distributions p is elliptical. For example, if X ˜p with mean vector μ and covariance Σ, then any linear projection a^{T}X is distributed according to p with mean μ=a^{T}μ and variance σ²=a^{T}Σa. An example is the multi-variate normal distribution, θₖ=(μₖ, Σₖ), with μₖ, Σₖ representing the mean vector and covariance matrix. In some embodiments, a similar reasoning applies to other distributions.

The joint distribution of the base ingredients is assumed independent and denoted collectively by the matrix of multivariate distributions,

P_{M,Σ}(X)=(p_{μ}_{₁}_{, Σ}_{₁}(x₁), . . . , p_{μ}_{ₙ}_{, Σ}_{ₙ}(xₙ))

where all the learned parameters are captured by the means matrix M=(μ₁, . . . , μₙ) and the covariance tensor Σ=(Σ₁, . . . , Σₙ). In some embodiments, the number of degrees of freedom can be reduced by asserting structure on the covariance Σ, such as Σₖ diagonal or low rank.

In some embodiments, due to ellipticity assumption, for any deterministic vector α, the mixture of the base ingredients is given by the distribution

P_{M,Σ}_{^{α}}=p_{Mα,Σα}_{²}

where Σα²=α₁²Σ₁+ . . . +αₙ²Σₙ.

For each measurement α, α′, Py, the two compared ingredients can be modeled as two independent random vectors X˜p_{Mα, Σα}_{²} and X′˜p_{Mα′, Σα′}_{²}, and their difference as the random vector ΔX˜p_{MΔα,ΣΔα}_{²}, where Δα=α−α′. A good embedding would make the subspace projection, PX˜p_{PMΔα, P(ΣΔα}_{²}_{)P}_{^{T}}, consistent with the measurements Py.

In some embodiments, this task can be carried out by defining the following negative log likelihood pointwise loss

$$\begin{matrix}{ℓ}_{i} ( M , Σ ) = - {log}_{{p}_{{P}_{i} M Δ {α}_{i} , {P}_{i} ( {Σ ( Δ {α}_{i} )}^{2} ) {P}_{i}^{T}}} ( {P}_{i} {y}_{i} ) \\ = {‖ {( M Δ {α}_{i} - y )}^{T} {{P}_{i}^{T} ( {P}_{i} ( {Σ ( Δ {α}_{i} )}^{2} ) {P}_{i}^{T} )}^{- 1} {P}_{i} ( M Δ {α}_{i} - y ) ‖}^{2}\end{matrix}$$

$$with {P}_{i} ( {Σ ( Δ {α}_{i} )}^{2} ) {P}_{i}^{T} = {( Δ {α}_{1} )}^{2} {P}_{i} {Σ}_{1} {P}_{i}^{T} + … + {( Δ {α}_{1} )}_{n}^{2} {P}_{i} {Σ}_{n} {P}_{i}^{T} ,$$

and solving the following (maximum likelihood) optimization problem

$${min}_{M , Σ} {\sum }_{i = 1}^{N} {ℓ}_{i} ( M , Σ ) .$$

In some embodiments, a Bayesian formulation is used, in which the posterior expectation of some loss function ρ(ΔX, y) is minimized. For example, in some embodiments, the correlation between ΔX and y in the subspace spanned by P, ρ(ΔX, y)=sign(ΔX)^{T}P^{T}Py, may be minimized.

In the latter case, a closed form expression exists of the sign of a normal variable, which is derived below for completeness. Let Z˜N(μ, σ²). Then,

$$P ( Z \leq 0 ) = Φ ( - \frac{μ}{σ} )$$

, where Φ denotes the cumulative density function of the normal distribution. Hence,

$$𝔼 \{ sign ( Z ) \} = 1 - 2 Φ ( - \frac{μ}{σ} ) .$$

Consequently, for measurement i, one embodiment can write the following pointwise loss:

$$\begin{matrix}{ℓ}_{i} ( M , Σ ) = 𝔼 {\{ sign ( {P}_{i} Δ X ) \}}^{T} {P}_{i} {y}_{i} \\ = ( 1 - 2 Φ ( \frac{- {p}_{i 1}^{T} M Δ {α}_{i}}{\sqrt{{p}_{i 1}^{T} ( ΣΔ {α}^{2} )} {p}_{i 1}} , … , \frac{- {p}_{i n}^{T} M Δ {α}_{i}}{\sqrt{{p}_{i n}^{T} ( ΣΔ {α}^{2} )} {p}_{i n}} ) ) {P}_{i} {y}_{i}\end{matrix}$$

(the function application is element-wise). As noted above, Bayesian loss minimization amounts to solving the following problem

$${min}_{M , Σ} {\sum }_{i = 1}^{N} {ℓ}_{i} ( M , Σ )$$

with the pointwise loss [symbol] ᵢ defined above.

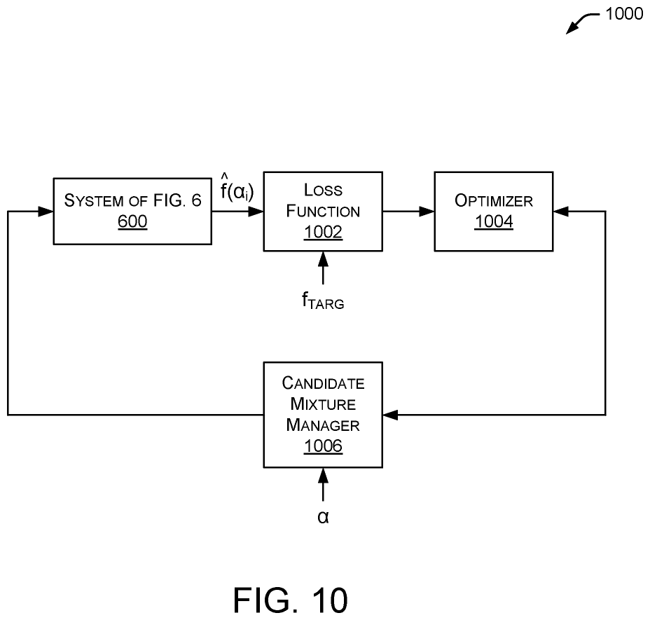

FIG. 10 is a block diagram illustrating an embodiment of a process flow 1000 for an inverse mode of operation. As shown in FIG. 10, the system 600 shown in FIG. 6 receives an initial candidate mixture coefficient α from a candidate mixture manager 1006. The system 600 generates a predicted feature \hat{f}ᵢ(αᵢ), which is provided to a loss function 1002. Loss function 1002 compares the predicted feature \hat{f}ᵢ(αᵢ) with the target result (e.g., a desired result based on taste, smell, texture, nutritional information, or the like). This comparison may include comparison of one or more features in the predicted feature \hat{f}ᵢ(αᵢ) with corresponding features in the target result. In some embodiments, loss function 1002 may be implemented using a pairwise comparator that compares the predicted feature \hat{f}ᵢ(αᵢ) with the target result.

The comparison result of the loss function 1002 (e.g., comparing the predicted feature \hat{f}ᵢ(αᵢ) with the target result) is provided to an optimizer 1004. Optimizer 1004 updates the candidate mixture definition based on the comparison result of the loss function 1002. The updated candidate mixture definition is then communicated to candidate mixture manager 1006, and the process is repeated iteratively.

The process flow 1000 optimizes the value of a while the parameters (θ) remain fixed. In some embodiments, optimizer 1004 tries different candidate mixture definitions until the predicted result matches (or is substantially close to) the target result based on taste, smell, texture, nutritional information, and the like.

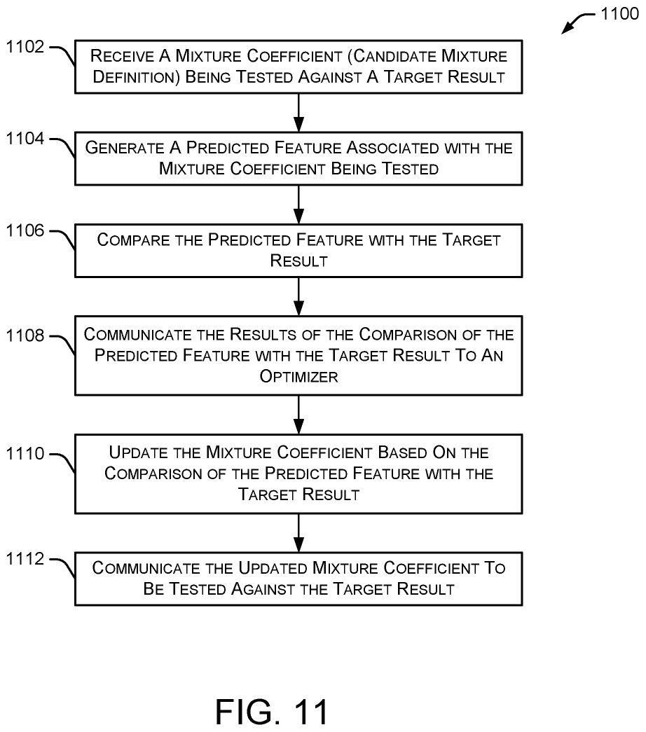

FIG. 11 is a flow diagram illustrating an embodiment of a process 1100 for implementing an inverse mode of operation. Initially, process 1100 receives 1102 a mixture coefficient (e.g., a candidate mixture definition) being tested against a target result. The process generates 1104 a predicted feature associated with the mixture coefficient being tested. The predicted feature is compared 1106 with the target result. Process 1100 communicates 1108 the results of the comparison of the predicted feature with the target result to an optimizer. The mixture coefficient is updated 1110 based on the comparison of the predicted feature with the target result. The updated mixture coefficient is communicated 1112 to an encoder (or other system or device) to be tested against the target result in an iterative manner.

After the embedding has been learned, the representation problem consists of finding the mixture coefficients a of the base ingredients that optimally describe another given ingredient. In some embodiments, the systems and methods are given another set of measurements comprising a set of pairwise comparisons Pᵢyᵢ of the target ingredient against mixtures of the base ingredients, each mixture represented by αᵢ (the comparisons are also possible with the new ingredient appearing in mixtures with base ingredients; however, for presentation clarity this discussion stays with the simpler formulation). Some embodiments aim at finding such a mixture of base ingredients β, that P_{M,Σ}β is maximally consistent with a set of measurements. The representation problem can be again considered as the minimization of one of the losses detailed above, this time with respect to β while keeping M,Σ fixed.

$${min}_{β ∈ {P}^{n}} {\sum }_{i = 1}^{N} {ℓ}_{i} ( β ) .$$

where Pⁿ={β:β≥0, β^{T}1=1} is the probability simplex, and [symbol] ᵢ is a pointwise loss.

In some embodiments, following the maximum likelihood formulation the pointwise negative log likelihood loss of the form

[symbol]

ᵢ(β)=−log p_{P,M(β-α}_{ᵢ}_{),P}_{ᵢ}_{(Σ(β-α}_{ᵢ}₎_{²}_{)P}_{ᵢ}_{^{T}}(Pᵢyᵢ).

is used.

In some other embodiments, following the Bayesian formulation, the pointwise loss of the form

$${ℓ}_{i} ( β ) = ( 1 - 2 Φ ( \frac{{p}_{i 1}^{T} M ( {α}_{i} - β )}{\sqrt{{p}_{i 1}^{T} ( {Σ ( β - {α}_{i} )}^{2} )} {p}_{i 1}} , … , \frac{{p}_{i n}^{T} M Δ ( {α}_{i} - β )}{\sqrt{{p}_{i n}^{T} ( {Σ ( β - {α}_{i} )}^{2} )} {p}_{i n}} ) ) {P}_{i} {y}_{i} .$$

is used.

Another version of the representation problem consists of approximating a base ingredient with a fixed subset of other base ingredients (e.g., replacing an animal ingredient with vegan ingredients). In some embodiments, this subset is denoted by restricting the mixtures to the subspace α=Qβ, where Q is a projection matrix. In this case, the distribution model, p_{μ}_{_{T}}, Σ_{T} of the target ingredient is known, and the approach aims at finding such a subset mixture, P_{M,Σ}Qβ that is closest to it in the sense of some divergence. This leads to the optimization problem

$${min}_{β ∈ {P}^{n}} D ( {P}_{M , Σ} Q β {‖}_{{p}_{{μ}_{T} , {Σ}_{T}}} ) ,$$

possibly with additional constraints on β such as sparsity. In some embodiments, the distance D is chosen to be the Kullback-Leibler divergence or the Wasserstein distance.

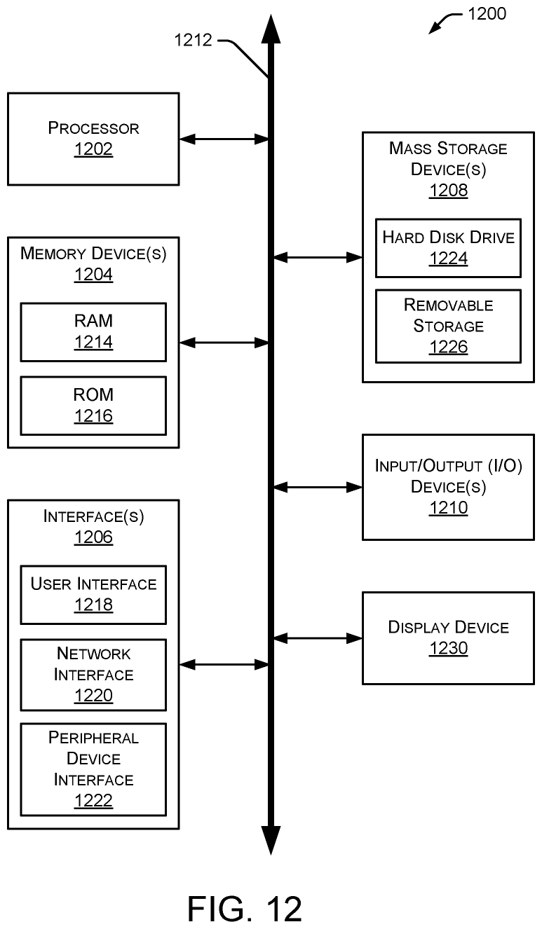

FIG. 12 illustrates an example block diagram of a computing device 1200 suitable for implementing the systems and methods described herein. In some embodiments, a cluster of computing devices interconnected by a communication network may be used to implement any one or more components of the systems discussed herein.

Computing device 1200 may be used to perform various procedures, such as those discussed herein. Computing device 1200 can function as a server, a client, or any other computing entity. Computing device can perform various functions as discussed herein, and can execute one or more application programs, such as the application programs described herein. Computing device 1200 can be any of a wide variety of computing devices, such as a desktop computer, a notebook computer, a server computer, a handheld computer, tablet computer and the like.

Computing device 1200 includes one or more processor(s) 1202, one or more memory device(s) 1204, one or more interface(s) 1206, one or more mass storage device(s) 1208, one or more Input/Output (I/O) device(s) 1210, and a display device 1230 all of which are coupled to a bus 1212. Processor(s) 1202 include one or more processors or controllers that execute instructions stored in memory device(s) 1204 and/or mass storage device(s) 1208. Processor(s) 1202 may also include various types of computer-readable media, such as cache memory.

Memory device(s) 1204 include various computer-readable media, such as volatile memory (e.g., random access memory (RAM) 1214) and/or nonvolatile memory (e.g., read-only memory (ROM) 1216). Memory device(s) 1204 may also include rewritable ROM, such as Flash memory.

Mass storage device(s) 1208 include various computer readable media, such as magnetic tapes, magnetic disks, optical disks, solid-state memory (e.g., Flash memory), and so forth. As shown in FIG. 12, a particular mass storage device is a hard disk drive 1224. Various drives may also be included in mass storage device(s) 1208 to enable reading from and/or writing to the various computer readable media. Mass storage device(s) 1208 include removable media 1226 and/or non-removable media.

I/O device(s) 1210 include various devices that allow data and/or other information to be input to or retrieved from computing device 1200. Example I/O device(s) 1210 include cursor control devices, keyboards, keypads, microphones, monitors or other display devices, speakers, printers, network interface cards, modems, lenses, CCDs or other image capture devices, and the like.

Display device 1230 includes any type of device capable of displaying information to one or more users of computing device 1200. Examples of display device 1230 include a monitor, display terminal, video projection device, and the like.

Interface(s) 1206 include various interfaces that allow computing device 1200 to interact with other systems, devices, or computing environments. Example interface(s) 1206 include any number of different network interfaces 1220, such as interfaces to local area networks (LANs), wide area networks (WANs), wireless networks, and the Internet. Other interface(s) include user interface 1218 and peripheral device interface 1222. The interface(s) 1206 may also include one or more user interface elements 1218. The interface(s) 1206 may also include one or more peripheral interfaces such as interfaces for printers, pointing devices (mice, track pad, etc.), keyboards, and the like.

Bus 1212 allows processor(s) 1202, memory device(s) 1204, interface(s) 1206, mass storage device(s) 1208, and I/O device(s) 1210 to communicate with one another, as well as other devices or components coupled to bus 1212. Bus 1212 represents one or more of several types of bus structures, such as a system bus, PCI bus, IEEE 1394 bus, USB bus, and so forth.

For purposes of illustration, programs and other executable program components are shown herein as discrete blocks, although it is understood that such programs and components may reside at various times in different storage components of computing device 1200, and are executed by processor(s) 1202. Alternatively, the systems and procedures described herein can be implemented in hardware, or a combination of hardware, software, and/or firmware. For example, one or more application specific integrated circuits (ASICs) can be programmed to carry out one or more of the systems and procedures described herein.

While various embodiments of the present disclosure are described herein, it should be understood that they are presented by way of example only, and not limitation. It will be apparent to persons skilled in the relevant art that various changes in form and detail can be made therein without departing from the spirit and scope of the disclosure. Thus, the breadth and scope of the present disclosure should not be limited by any of the described exemplary embodiments, but should be defined only in accordance with the following claims and their equivalents. The description herein is presented for the purposes of illustration and description. It is not intended to be exhaustive or to limit the disclosure to the precise form disclosed. Many modifications and variations are possible in light of the disclosed teaching. Further, it should be noted that any or all of the alternate implementations discussed herein may be used in any combination desired to form additional hybrid implementations of the disclosure.

1. An apparatus comprising:
an encoder receiving a plurality of base ingredients and producing a plurality of corresponding representations;
a composite modeler coupled to the encoder and configured to receive a mixture definition comprising a list of base ingredients and their relative proportions, and output a representation of the mixture definition; and
a decoder coupled to the composite modeler and configured to receive a representation of a mixture and output a list of features.

2. The apparatus of claim 1, wherein each feature in the list of features includes a numerical value labeled by the feature name.

3. The apparatus of claim 2, wherein the list of features is a vector having dimensions that are annotated by the feature names.

4. The apparatus of claim 1, wherein the list of features includes at least one of a taste, a smell, a texture, or a nutritional value.

5. The apparatus of claim 1 further comprising a pairwise comparator coupled to the decoder and configured to receive a pair of lists of features and produce a list of pairwise comparisons.

6. The apparatus of claim 5, wherein the pairwise comparator is further configured to determine if one of the mixtures has a stronger presence of the feature than the other mixture.

7. An apparatus comprising:
an encoder receiving a plurality of base ingredients and producing a plurality of corresponding representations;
a composite modeler coupled to the encoder and configured to receive a mixture definition comprising a list of base ingredients and their relative proportions, and output a representation of the mixture;
a decoder coupled to the composite modeler and configured to receive a representation of a mixture and output a list of features;
a loss function configured to receive a plurality of training mixture definitions and a plurality of training pairwise comparisons, and produce a number based on the plurality of training pairwise comparisons; and
an optimizer configured to adjust a plurality of parameters of the system to minimize the value of the loss function.

8. The apparatus of claim 7, further configured to:
receive the plurality of training mixture definitions;
output a corresponding plurality of pairwise comparisons to the loss function based on the plurality of training mixture definitions; and
quantify, using the loss function, the agreement of the said pairwise comparisons to the corresponding training pairwise comparisons.

9. The apparatus of claim 7, wherein the number produced based on the plurality of training pairwise comparisons predicts whether a particular feature is stronger in one of the compared mixture definitions.

10. The apparatus of claim 7, wherein the loss function is further configured to receive ground truth information associated with the pairwise comparisons.

11. The apparatus of claim 10, wherein the ground truth information is generated based on at least one of human tasting or mechanical properties.

12. The apparatus of claim 7, wherein the optimizer is further configured to provide the adjusted parameters to the encoder.

13. An apparatus comprising:
an encoder receiving a plurality of base ingredients and producing a plurality of corresponding representations;
a composite modeler coupled to the encoder and configured to receive a mixture definition comprising a list of base ingredients and their relative proportions, and output a representation of the mixture;
a decoder coupled to the composite modeler and configured to receive a representation of a mixture and output a list of features;
a candidate mixture definition manager configured to receive a candidate mixture definition and produce a corresponding list of features;
a loss function configured to receive a target list of features and produce a number; and
an optimizer coupled to the candidate mixture definition manager and configured to update the candidate mixture definition to minimize the value of the loss function.

14. The apparatus of claim 13, wherein the loss function is configured to quantify an agreement of the list of features produced based on the target list of features.

15. The apparatus of claim 13, wherein the loss function is further configured to produce a number based on a similarity between the target list of features and the candidate mixture definition.

16. The apparatus of claim 13, wherein the loss function includes a pairwise comparator configured to compare a predicted feature to the target list of features.

17. The apparatus of claim 13, wherein the optimizer is further configured to provide the updated candidate mixture definition to the encoder.

18. A method comprising:
receiving ingredient data associated with a plurality of base ingredients;
producing a plurality of representations corresponding to the plurality of base ingredients;
receiving a mixture definition comprising a list of base ingredients and their relative proportions;
generating an output representation of the mixture definition;
receiving a representation of a mixture; and
generating an output list of features of the mixture.

19. The method of claim 18, further comprising:
receiving a plurality of training mixture definitions;
receiving a plurality of training pairwise comparisons;
generating a number based on the plurality of training pairwise comparisons; and
adjusting a plurality of parameters to minimize a value of a loss function.

20. The method of claim 18, further comprising:
receiving a candidate mixture definition;
generating a corresponding list of features associated with the candidate mixture definition;
receiving a target list of features;
generating a number based on the target list of features; and
updating the candidate mixture definition to minimize a value of a loss function.
